(19) United States
(12) Patent Application Publication
(10) Pub. No.: US 2026/0283965 A1
(43) Pub. Date: Sep. 24, 2026

(54) PHARMACEUTICALLY ACTIVE COMPOSITIONS AND USES THEREOF

(71) Applicant: XERIENT PHARMA LTD., Petah Tikva (IL)

(72) Inventors: Michael Shtein, Kfar Daniel (IL); Guy Yachin, Potomac, MD (US); Galia Temtsin-Krayz, Ashdod (IL); Dannit Licht, Givat-Shemuel (IL); Pavel Kazhdan, Sderot (IL); Ofer Doron, Kibbutz Lahav (IL)

(21) Appl. No.: 19/472,948

(22) PCT Filed: Apr. 4, 2024
(86) PCT No.: PCT/US2024/023104

§ 371 Date: Oct. 6, 2025

Related U.S. Application Data
(60) Provisional application No. 63/456,842, filed on Apr. 4, 2023.

(51) Int. Cl. A61K 9/50; A61K 31/4415; A61K 31/4985; A61K 31/661

(52) U.S. Cl. CPC A61K 9/5015; A61K 31/4415; A61K 31/4985; A61K 31/661

(57) ABSTRACT

The disclosure relates to pharmaceutical compositions of active pharmaceutical ingredients (API), for example—cytoprotective pro-drugs and uses thereof. More specifically, the disclosure is related to compositions, methods and uses of cytoprotective pro-drug microcapsules in a dispersion, or as micro-pellets, micro-beadles, micro-granules or mini tablets, all configured to activate upon exposure to a triggering event.

### BACKGROUND

The disclosure is directed to pharmaceutical compositions of cytoprotective pro-drugs and uses thereof. More specifically, the disclosure is related to compositions, methods and uses of cytoprotective pro-drug microcapsules in a dispersion, or as micro-pellets, micro-beadles, micro-granules or mini tablets, all configured to activate upon exposure to a triggering event.

The major limiting factor in delivering the appropriate tumoricidal dose of radiation is toxicity to normal tissue in adjacent organs. This issue is highlighted by solid tumors of the abdomen and pelvis, such as colorectal and prostate adenocarcinoma, which often cannot achieve tumoricidal doses without significant morbidity to the gastrointestinal (GI) tract. Moreover, radiation therapy of pelvic malignancies, frequently results in dose-limiting toxicities such as serous, mucoid, or more rarely, bloody diarrhea.

Other dose-limiting toxicity is reported for other cancer types, such as esophageal cancer, ovarian cancer, and cervical cancer.

However, in order to ensure the therapeutically effective accretion of the cytoprotective API in the tissue adjacent to the radiated organ prior to the commencement of radiation, it is necessary to increase the stability of the cytoprotective API, ensuring optimal efficiency at the protection site.

Prodrugs can offer many advantages over parent drugs such as increased solubility, enhanced stability, improved bioavailability, reduced side effects, and better selectivity. Specifically, cytoprotective pro-drugs designed for topical activation at the protection site, can offer an advantage over other dosage forms.

### SUMMARY

In an exemplary implementation, provided is a pharmaceutical dispersion comprising: a dispersed phase comprising: a plurality of controlled-release microcapsules, micro-pellets, micro-beadles, micro-granules or mini tablets comprising a cytoprotective pro-drug or an active pharmaceutical agent (API), and an activating agent; and a continuous phase comprising a hydrocolloid matrix configured, upon exposure to a predetermined event, to allow contact between the plurality of controlled-release microcapsules comprising the cytoprotective pro-drug or the API, and the activating agent.

In another exemplary implementation, provided herein is use of a pharmaceutical dispersion comprising: a dispersed phase comprising: a plurality of controlled-release microcapsules, micro-pellets, micro-beadles, micro-granules or mini tablets comprising a cytoprotective pro-drug or an active pharmaceutical agent (API), and an activating agent; and a continuous phase comprising a hydrocolloid matrix configured, upon exposure to a predetermined event, to allow contact between the plurality of controlled-release microcapsules comprising the cytoprotective pro-drug or the API, and the activating agent, in the process irradiating a tumor associated with: colorectal cancer, prostatic cancer, ovarian cancer, cervical cancer, or esophageal cancer.

The microcapsules can be a part of a delivery functional gel and used “as is”, or can be formed upon exposure of regular tablets, capsules or mini tablets to GI fluids.

### BRIEF DESCRIPTION OF THE DRAWINGS

The features of the compositions, methods and uses of cytoprotective pro-drug microcapsules in a dispersion, micro-pellets, micro-beadles, micro-granules or mini tablets and their combination, configured to activate upon exposure to a triggering event, will become apparent from the following detailed description when read in conjunction with the drawings, which are exemplary, not limiting, are depicted in several figures and in which:

FIG. 1, depicts Mini and regular tablets; and

FIG. 2, depicts Different sized micro and mini capsules in gel.

While the disclosure is amenable to various modifications and alternative forms, specifics thereof have been shown by way of example in the figures and will be further described in detail hereinbelow. It should be understood, however, that the intention is not to limit the disclosure to the particular exemplary implementations described. On the contrary, the intention is to cover all modifications, equivalents, and alternatives.

### DETAILED DESCRIPTION

The disclosure relates in one exemplary implementation to compositions, methods and uses of cytoprotective pro-drug microcapsules in a dispersion configured to activate upon exposure to a triggering event.

Close proximity of pelvic tumors (prostate, bladder, colorectal or gynecological; e.g., ovarian, cervical) to the anterior rectal wall makes it impossible for the rectal wall to avoid exposure to radiation during therapy involving tumoricydic radiation dosage. The resulting toxicity (e.g., mucositis) becomes dose limiting and therefore affecting outcomes. Similarly, thoracic and head and neck cancer radiation therapy can cause damage to nearby healthy organs such as the esophagus, causing acute radiation-induced esophageal damage (ARIED). Furthermore, acute radiation-induced esophageal damage (ARIED) is often a dose limiting factor during lung and other head and neck cancers, including esophageal cancer.

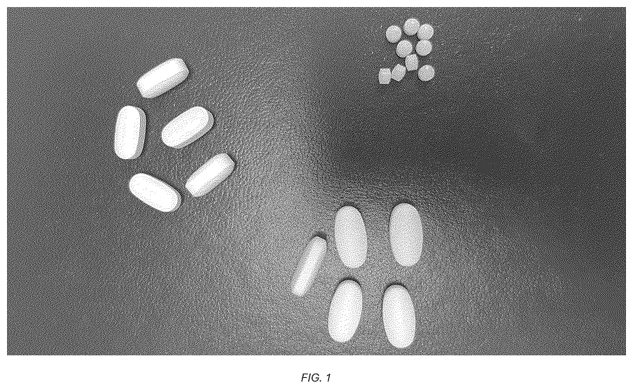

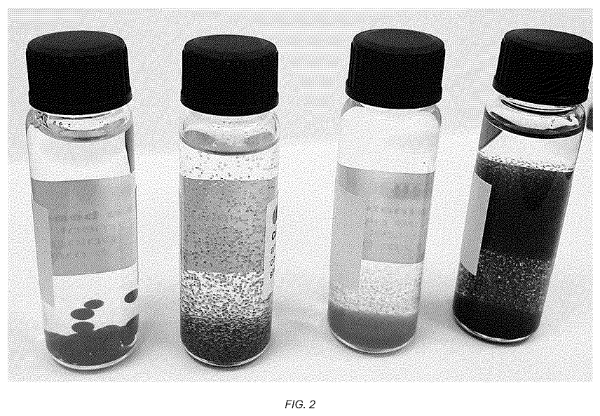

Accordingly and in an exemplary implementation, provided herein is a pharmaceutical dispersion comprising: a dispersed phase comprising: a plurality of controlled-release microcapsules (see e.g., FIG. 2), micro-pellets, micro-beadles (see e.g., FIG. 1), micro-granules, mini tablets or a combination thereof comprising a pharmaceutically effective amount of at least one cytoprotective pro-drug or an active pharmaceutical agent, and an activating agent; and a continuous phase comprising a hydrocolloid matrix configured, upon exposure to a predetermined event, to allow contact between the plurality of controlled-release microcapsules comprising the cytoprotective pro-drug or the active pharmaceutical agent, and the activating agent.

In the context of the disclosure, microparticles, nanoparticles, granules, pellets, and/or minitablets, which typically lack wall coating, serve as subunits. These subunits, primarily comprising a multitude of small discrete units, are compressed into tablet form or enclosed in a sachet, gel, or capsule to facilitate delivery of the recommended total dose. For example, the micro-pellets used herein can be a pellet or particle of a pharmaceutical composition which may have any shape, such as discs and cylinders. Such discs or cylinders will typically have diameters generally in the range of 250 μm to 750 μm. Other shapes may also include for instance oblong, spheroidal, and the like. It will be understood by those of ordinary skill in the art that generally any shape will be acceptable with the understanding that the volume of such particle shapes be substantially similar to the volume of a sphere described by the above diameter ranges.

Furthermore, in the context of the disclosure and claims, the term “controlled release” refers to any composition which comprising a pharmaceutically effective amount of at least one cytoprotective pro-drug or an active pharmaceutical agent, which is formulated to provide a gradual release of the pharmaceutically effective amount of at least one cytoprotective pro-drug or an active pharmaceutical agent, over a predetermined period of time so that the concentration is maintained in the target site for that time period at a uniform concentration and also refer to immediate release pharmaceutical composition or is exchangeable with for example, extended release, sustained release, delayed release, or pulsed-release at a particular time. The rate controlling agents may be polymers, which are insoluble in water but semi permeable or permeable to physiological fluids.

The term “pharmaceutical dispersion” refer in an exemplary implementation to a system of at least two phases, wherein one of these phases is the continuous phase and at least one phase is dispersed. Dispersion is a generic term, which encompasses e.g. emulsions, wherein the continuous phase is liquid and the dispersed phase is liquid, suspensions wherein the continuous phase is liquid and the dispersed phase is solid, solid aerosole, wherein continuous phase is gas and the dispersed phase is solid, in other words, a suspension, emulsion, solution, gel, or a duplex continuous system such as oil-in-water-in-oil (OWO) emulsion, or water-in-oil-in-water (WOW) emulsion, or a combination of continuous systems.

Moreover, the term “cytoprotective” refers to the capacity of agents, e.g. chemical compounds, either natural or not, of maintaining interactions of the cells with each other or with other tissues, of protecting the cells against degeneration phenomena leading to cell function loss or to undesirable cell activities, with or without cell death, and/or against cell dysfunctions and/or against degenerative diseases or affections leading to the cell dysfunctions, said dysfunctions or said diseases or affections either leading to cell death or not.

In an exemplary implementation, the cytoprotective pro-drug and/or activating, or active pharmaceutical agent (API) used in the continuous system pharmaceutical composition disclosed, comprises: ergotamine, and/or amifostine, and/or pyridoxine.

In an exemplary implementation, the cytoprotective pro-drug, or API is an organic thiophosphate amifostine. Dephosphorylation of amifostine at the cell membrane by alkaline phosphatase releases the free thiol WR-1065. The comparative hypovascularity of tumors leads to tissue hypoxia, anaerobic metabolism, and a low interstitial pH. The lower pH, coupled with higher alkaline phosphatase levels in normal tissues and capillaries and arterioles results in higher alkaline phosphatase activation of the prodrug in normal tissues than tumors. WR-1065 can bind directly to the active species of chemotherapeutic agents or ionizing radiation, within normal cells, resulting in their deactivation. Thus, selective protection of normal tissues is the result of reduced metabolism of amifostine to WR-1065 and low uptake of WR-1065 by tumors. The prodrug S-2-(3-aminopropylamino)ethyl dihydrogen phosphorothioate (hereinafter WR-2721, or amifostine) is represented by the formula:

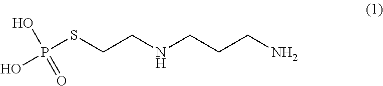 [SMILES: NCCCNCCSP(=O)(O)O]

It is noted, that the pro-drug API includes also its free mono-base or di-base conjugate, devoid of the respective HCl and any other pharmaceutically acceptable salt formation, as well as metabolite having the formula:

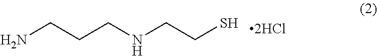

In another exemplary implementation, the pharmaceutically effective amount of at least one cytoprotective pro-drug or an active pharmaceutical agent used, is ergotamine, or pyridoxine, their combination, pro-drug thereof and conjugates thereof.

An “effective amount” of a subject compound, with respect to the pharmaceutical compositions, methods and uses, refers to an amount of the cytoprotective pro-drug in a preparation which, when applied as part of a desired dosage regimen (dose, formulation, frequency), prevents from bringing about, e.g., a negative change in rate of survival of a cell according to clinically acceptable standards, in other words, having therapeutic efficacy. Likewise, “therapeutic efficacy” denotes the capacity of a substance to achieve a certain therapeutic effect, e.g. reduction in cell death as result of irradiation. Therapeutic effects can be measured by determining the extent in which a substance can achieve the desired effect, typically in comparison with another substance under the same circumstances. A suitable measure for the therapeutic efficacy is the ED₅₀ value, which may for example be determined during clinical trials or by plasma exposure tests.

In an exemplary implementation, the API pro-drug is WR-2721. As used herein, the term “pro-drug” refers to a pharmacologically inactive form of a compound that undergoes biotransformation prior to exhibiting its pharmacological effect(s). A pro-drug is one that is converted in vivo by a subject after administration into a pharmacologically active form of the compound in order to produce the desired pharmacological effect. After administration to the subject, the pharmacologically inactive form of the compound is converted in vivo under the influence of biological fluids and/or enzymes and/or other activating agents into a pharmacologically active form of the compound. Pro-drug forms of compounds can be utilized, for example, to improve bioavailability, mask unpleasant characteristics such as bitter taste, alter solubility for intravenous use, or to provide site-specific delivery of the compound. Reference to a compound herein includes pro-drug forms of a compound and the drug conjugate (active form). In another exemplary implementation, the activating agent used in the dispersed phase of the suspended systems forming the pharmaceutical compositions disclosed (referring to a substance or compound that initiates or facilitates a specific biochemical reaction or process), can be, for example: a protease, and/or Oxyreductase, and/or Carboxylesterase, and/or Paraoxonase, and/or Alkaline phosphatase, and/or b-glucuronidase, and/or Cytosine deaminase. For example, members of the serine hydrolase superfamily such as Carboxylesterases can hydrolyze a variety of ester-, amide-, and carbamate-containing API's to their respective free acids. They act as an effective biological barrier to limit the distribution of substrates that might be toxic and facilitate their elimination by turning them into more polar molecules. Additionally, or alternatively, oxyreductases, such as, for example, cytochrome P450. The local concentrations (of the over 50 forms) differ for the various P450 enzymes. Accordingly, in an exemplary implementation, an activating cytochrome P450 enzymes used as the activating agent, is configured to selectively target drug activation to specific tissues. The oxyreductase can insert an oxygen into a C—H or N—H bond to give the corresponding hydroxyl derivative, and/or add an oxygen atom to the electron pair of a nitrogen or sulfur atom, resulting in the formation of an N-oxide or S-oxide, and/or catalyze the epoxidation of olefinic or aromatic π-bonds. Likewise, paraoxonase, is a family of hydrolases, such as esterase, which can catalyze the hydrolisys of various arylesterases and organophosphates (e.g., amifostine).

As indicated, the cytoprotective pro-drug or API is incorporated as a microcapsule within the continuous phase matrix. In the context of the disclosure, the term “microcapsules” refer generally to tiny particles or droplets of spherical objects, which consist of a core and a wall material surrounding the core, wherein the core in principal can be a solid, liquid or gaseous component, which is surrounded by the solid wall material. For many applications, the wall is formed by a polymer material. Microcapsules usually have a volume average diameter from 1 μm to 1000 μm.

A multitude of shell materials can be used in certain exemplary implementations to form the wall of microcapsules. The shell can consist either of natural, semisynthetic or synthetic materials. Natural shell materials are, for example, gum arabic, agar agar, agarose, maltodextrins, alginic acid or its salts, e.g. sodium alginate or calcium alginate, fats and fatty acids, cetyl alcohol, collagen, chitosan, lecithins, gelatin, albumin, shellac, polysaccharides, such as starch or dextran, polypeptides, protein hydrolyzates, sucrose and waxes and a combination comprising one or more of the foregoing. Semisynthetic shell materials are, for example; chemically modified celluloses, in particular cellulose esters and cellulose ethers, e.g. cellulose acetate, ethyl cellulose, hydroxypropylcellulose (HPC), hydroxypropylmethylcellulose (HPMC) and carboxymethyl-cellulose (CMC), and also starch derivatives, in particular starch ethers and starch esters. Synthetic shell materials are, for example, polymers, such as polyacrylates, polyamides, polyvinyl alcohols, polyvinylpyrrolidones or polyureas and their copolymers and terpolymers. Depending on the type of shell material and the production process, microcapsules are formed in each case with different properties, such as diameter, size distribution and physical and/or chemical properties.

The wall material of the microcapsules used in the methods and compositions disclosed can be, for example polymers that can be dissolved at a predetermined pH value. The polymer can be, for example poly(methacrylic acid-co-ethylacrylate, poly(methacylilc acid-co-methyl methacrylate), poly(methyl acrylate-co-methylmethacrylate-co-methacrylic acid) 7:3, cellulose acetate phthalate, cellulose acetate succinate, methyl cellulose phthalate, methylhydroxy propylcellulose phthalate, ethyl hydroxycellulose phthalate, poly(vinylacetate phthalate, polyvinylbutyrate acetate, vinyl acetate-maleic anhydride copolymer, styrene-maleic mono-ester copolymer, methyl aerylate-methacrylic acid copolymer, and methacrylate-methacrylic acid-octyl acrylate copolymer, and/or enteric polymers such as cellulose acetate phthalate, cellulose acetate succinate, methylcellulose phthalate, ethylhydroxycellulose phthalate, polyvinylacetatephthalate, styrene-maleic mono-ester copolymer, methacrylic acid methyl methacrylate copolymer methyl acrylate-methacrylic acid copolymer, methacrylate-methacrylic acid-octyl acrylate copolymer, etc. These may be used either alone or in combination, or together with other polymers than those mentioned above. The enteric coating may also include insoluble substances which are neither decomposed nor solubilized in living bodies, such as alkyl cellulose derivatives such as ethyl cellulose, crosslinked polymers such as styrene-divinylbenzene copolymer, polysaccharides having hydroxyl groups such as dextran, cellulose derivatives which are treated with bifunctional crosslinking agents such as epichlorohydrin, dichlorohydrin, 1, 2-, 3, 4-diepoxybutane, etc. The enteric coating may also include starch and/or dextrin. For example, the enteric-coating materials are the pharmaceutically acceptable methacrylic acid copolymer which are copolymers, anionic in character, based on methacrylic acid and methyl methacrylate, for example having a ratio of free carboxyl groups: methyl-esterified carboxyl groups of 1:>3, e.g. around 1:1 or 1:2, and with a mean molecular weight of 135,000.

Such polymers are sold under the trade name Eudragit™, such as the Eudragit L series e.g. Eudragit L 12.5™, Eudragit L 12.5P™, Eudragit L100™, Eudragit L 100-55™, Eudragit L-30™, Eudragit L-30 D-55™, the Eudragit S™ series e.g. Eudragit S 12.5, Eudragit S 12.5P™, Eudragit S100™, the Eudragit NE™ series e.g. Eudragit NE 30D™, the Eudragit RL™ series, e.g. Eudragit RL 12.5™, Eudragit RL 100™, Eudragit RL PO™, Eudragit RL 30D™, and the Eudragit RS™ series e.g. Eudragit RS 12.5™, Eudragit RS 100™, Eudragit RS PO™, and Eudragit RS 30D™ Furthermore, some of these polymers are known and sold as enteric polymers, for example having a solubility in aqueous media at pH 5.5 and above, such as the commercially available “Eudragit” (Trade Mark) enteric polymers, such as “Eudragit L100-55” (Trade Mark) i.e. a cationic polymer synthesised from dimethylaminoethyl methacrylate.

Moreover, the polymers forming the wall material of the microcapsules may be used either alone or with a plasticizer. Such coatings are normally applied using a liquid medium, and the nature of the plasticizer depends upon whether the medium is aqueous or non-aqueous. Aqueous plasticizers include propylene glycol or “Citroflex” or Citroflex A2” (Trade Marks) (mainly triethyl citrate or acetyl triethyl citrate). Non-aqueous plasticizers include these, and also diethyl and dibutyl phthalate and dibutyl sebacate. The quantity of plasticizers included will be apparent to those skilled in the art. The enteric coating may also include an anti-tack agent such as talc, silica or glyceryl monostearate. The quantity of plasticizers and anti-tack agent may be generally conventional to the art. Typically the coating may include around 10-40 wt. % plasticiser and up to around 50 wt % of anti-tack agent, e.g. 2-20 wt. % of anti-tack agent.

Actual dosage levels of the active ingredients in the pharmaceutical compositions provided herein, can be varied so as to obtain an amount of the active ingredient or the prodrug (e.g., WR-2721) that is effective in achieving the desired therapeutic response for a particular patient, composition, and mode of administration, without being toxic to the patient. The selected dosage level will depend upon a variety of factors including the activity of the particular compound provided herein, which can employed, or the ester, salt or amide thereof, the dosage form, e.g., a suppository, a subligual tab, etc., the route of administration, the time of administration, the rate of excretion or metabolism of the particular cytoprotective pro-drug and activating agent being employed, the rate and extent of absorption, the duration of the treatment, other drugs compounds (e.g., oxaliplatin, irinotecan, fluorouracil, leucovorin, gemcitabine in the case of metastatic prostate cancer), and/or materials used in combination with the particular compound employed, the age, sex, weight, condition, general health and prior medical history of the patient being treated, and like factors and their combination.

In certain exemplary implementations, the suspended pharmaceutical composition is provided in a suppository form. In the context of the disclosure, the term “suppository” refers to dosage forms or formulations that are inserted into body cavities. Suppositories are solid bodies of various sizes and shape, which dissolve, disintegrate or melt upon administration. Suppositories can be provided in a variety of shapes and sizes. In certain exemplary implementations, vaginal suppositories are suitable for insertion into a vaginal cavity or urogenital tract, with or without an applicator. For example, vaginal suppositories can be inserted with an applicator high into the vaginal tract or with or without an applicator into the vaginal cavity. Vaginal suppositories are usually globular, oviform, or cone-shaped, but can be of any shape or size suitable for insertion into the vaginal cavity. A tablet, a ring or a capsule suitable for insertion into vaginal cavity or urogenital tract is included in the scope of the term “suppository. These suppositories can be used, in certain exemplary implementations, to provide a cytoprotective pro-drug and an activating agent (e.g., enzyme) in the course of radiation for cervical cancer, and/or ovarian cancer. For example, carriers for the suspended pharmaceutical compositions disclosed herein in suppository form can be: cocoa butter (refers to mixtures of triglycerides of saturated and unsaturated fatty acids, such as stearic, palmitic, oleic, lauric, and linoleic acids), a cocoa butter substitute (refers to vegetable oils modified by esterification, hydrogenation, etc., including hydrogenated vegetable oil and hard fat), glycerinated gelatin, a polyethylene glycol-based carrier, and a surfactant (e.g., polyoxyethylene sorbitan fatty-acid esters and polyoxyethylene stearates) or a combination comprising one or more of the foregoing.

In certain exemplary implementations, the suspended pharmaceutical composition is provided in a form of a tab, or esophageal tab (using e.g., an applicator), configured for trans-mucosal delivery of the cytoprotective pro-drug microcapsules. Mouth dissolving films, or orodispersible films (ODF), consist in certain exemplary implementations, of a very thin strip of a given size with a known payload of the cytoprotective pro-drug and the activating agent (the “Tab”), which is placed for example on the tongue, under the tongue, or in the throat of a user in need thereof, or any oral mucosal tissue, then, wet by saliva, leading to rapid disintegration and release of the entrapped ingredient for oromucosal absorption, or, with formula modifications, will maintain the quick-dissolving aspects allow for the cytoprotective pro-drug microcapsules to dissolve and reaction between the activating agent and the cytoprotective pro-drug to be achieved.

For example, the tab can be thin (in other words, less than 4 mm) film with an area of 5-20 cm² containing cytoprotective pro-drug (e.g., amifostine) microcapsules and an activating agent (e.g., PK). The dissolution in one embodiment, (or disintegration, breakdown, fragmentation, degeneration, deagglomeration or any other process leading to immediate, controlled or sustained release or delivery of the cytoprotective pro-drug microcapsules and the activating agent), in water or saliva respectively, is reached through a special matrix fabricated from polymers that are at least partially water-soluble. The cytoprotective pro-drug (e.g., amifostine) microcapsules and an activating agent (e.g., PK) composition can be incorporated up to any single dose that, due to the particular API used, will not adversely affect the integrity of the tab and its ability to be physically manipulated by a user, nor affect the physico-chemical properties of the carrier matrix. For example, cytoprotective pro-drug concentration of between about 100 mg at a loading range of between about 0.1% to about 50% (w/w) of the carrier (matrix).

External formulation considerations (e.g., plasticizers, crosslinking agents, etc.) can be made to affect mechanical properties of the films, such as, for example, shifting the glass transition temperature (Tg) of the matrix forming the tab to a temperature below the mouth (or room) temperature, flow modifiers, surfactants and the like that will affect the mechanical, chemical and biological properties of the resulting tabs, as well as their adhesive properties for a better targeted delivery.

Likewise, the suspended pharmaceutical composition can be delivered in an oral form, configured for burst release of the cytoprotective pro-drug microcapsules and the activating agent. The oral dosage forms can be also be a part of a composition comprising salt of a chelating agent selected from the group consisting of EDTA, EGTA, citrate and therapeutically acceptable salts thereof. Furthermore, these compositions may also contain excipients such as fillers disintegrant, flowing agent, gliadents, binders, wetting agents, lubricants, and/or antioxidants addition, the oral compositions provided herein may also comprise an acceptable buffering agent mixture (e.g., tris buffer, phosphate buffer, and the like); a sweetening agent; and/or at least one flavor agent.

In an exemplary implementation, the predetermined event, or trigger configured to release the microcapsules of the cytoprotective pro-drug and the activating agent from the dosage form disclosed herein is exposure to a temperature of between about 30° C. and about 44° C. for a period of between about 30 seconds and about 30 min. The release can be controlled, sustained or immediate, and be configured by altering the wall material on the microcapsules, as well as the microcapsules' size and size distribution. In another exemplary implementation, the predetermined event is exposure to a temperature of between about 36° C. and about 44° C. for a period of between about 0.5 minutes (min.) and about 30 min., at pH of between about 4.0 and about 7.8 and the cytoprotective pro-drug or the API is configured to have site-specific activity following activation by the activating agent.

In the context of the disclosure, the term “site-specific” refers to the targeted action or localization of a drug or active pharmaceutical ingredient (API) within a particular region or site in the body, such as the gastrointestinal (GI) tract. Site-specificity involves the design of drugs or formulations in such a way that they exert their therapeutic effects primarily at the intended site of action while minimizing systemic distribution and potential side effects in other parts of the body. This is achieved in certain exemplary implementations, through various means, including specific drug delivery systems (DDS), formulations, or chemical modifications that enhance targeting or retention at the desired site. Additionally, site-specificity can also refer to the molecular or cellular level, where drugs are designed to interact with specific targets, such as enzymes, receptors, or cell types, present within the GI mucosa or associated structures.

Accordingly, the Active pharmaceutical ingredients (APIs) are configured to exhibit various site-specific activities within the targeted gastrointestinal (GI) tract location, contributing to their therapeutic efficacy and minimizing systemic side effects. These can be, for example, local absorption enhancement, where APIs or co-administered drugs are formulated to target specific regions of the GI tract, such as the duodenum and jejunum, to promote permeability or inhibit efflux transporters, thereby enhancing absorption. Additionally, APIs are configured in another implementation, to modulate enzymes involved in digestion, such as lipases, proteases, or carbohydrases, thereby influencing metabolic pathways of co-drugs used. Furthermore, certain APIs are configured to act locally to reduce inflammation associated with radiation treatment, targeting specific inflammatory mediators or pathways within the GI mucosa. By “treatment” is meant, in the context of the disclosure, a treatment that is preventive, advantageously in an individual at risk, and/or curative and/or palliative. It includes the fact of inhibiting and/or eliminating the appearance and/or the development of a pathological situation or of a degenerative process or of reducing the severity of such pathological situation or such degenerative process, such as reducing the number or severity of symptoms associated with the pathological situation or the degenerative process, enhancing the quality of life of the individual suffering from such pathological situation or such degenerative process, decreasing the amounts of other medications needed to treat the pathological situation or the degenerative process, enhancing the effect of another treatment taken to treat the pathological situation or the degenerative process, or prolong the life of individuals suffering from the pathological situation or the degenerative process.

In an exemplary implementation, the compositions disclosed are used in the methods described. Accordingly, provided herein is a use of the pharmaceutical dispersion as disclosed herein, in the process irradiating a tumor associated with: colorectal cancer, prostatic cancer, ovarian cancer, cervical cancer, or esophageal cancer.

In the context of the disclosure, the term “pharmaceutically accepted” salt means a salt that is acceptable for administration to a patient, such as a mammal (salts with counter ions having acceptable mammalian safety for a given dosage regime). Such salts may be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions known in the art and include, for example, sodium, potassium, calcium, magnesium, ammonium, tetraalkylammonium, etc., and when the molecule contains a basic functionality, salts of organic or inorganic acids, such as hydrochloride, hydrobromide, formate, tartrate, besylate, mesylate, acetate, maleate, oxalate, etc.

The terms “first,” “second,” and the like, herein do not denote any order, quantity, or importance, but rather are used to denote one element from another. The terms “a”, “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the tumor(s) includes one or more tumor). Reference throughout the specification to “one exemplary implementation”, “another exemplary implementation”, “an exemplary implementation”, and so forth, means that a particular element (e.g., feature, structure, and/or characteristic) described in connection with the exemplary implementation is included in at least one exemplary implementation described herein, and may or may not be present in other exemplary implementations. In addition, it is to be understood that the described elements may be combined in any suitable manner in the various exemplary implementations.

Accordingly and in an exemplary implementation, provided herein is a pharmaceutical dispersion comprising: a dispersed phase comprising: a plurality of controlled-release microcapsules, micro-pellets, micro-beadles, micro-granules or mini tablets comprising a cytoprotective pro-drug or an active pharmaceutical agent (API), and an activating agent; and a continuous phase comprising a hydrocolloid matrix configured, upon exposure to a predetermined event, to allow contact between the plurality of controlled-release microcapsules comprising the cytoprotective pro-drug or the API, and the activating agent, wherein (i) the cytoprotective pro-drug or active pharmaceutical agent comprises: ergotamine, amifostine, pyridoxine, or a composition comprising one or more of the foregoing and any permutation thereof, (ii) the cytoprotective pro-drug or active pharmaceutical agent comprises: S-2-(3-amino propylamino)ethyl dihydrogen phosphorothioate, its active 2-[(3-Aminopropyl) amino]ethanethiol dihydrochloride (WR-1065), their combination (at any permutation) and their pharmaceutically accepted salt, wherein (iii) the activating agent is: a protease, Oxyreductase, Carboxylesterase, Paraoxonase, Alkaline phosphatase, β-glucuronidase, Cytosine deaminase, or an activating agent composition comprising one or more of the foregoing, wherein (iv) the pharmaceutical dispersion is in a suppository form, (v) in an injectable form, (vi) in an oral tab form, wherein (vii) the predetermined event is exposure to a temperature of between about 36° C. and about 44° C. for a period of between about 3 minutes (min.) and about 30 min, or (viii) the predetermined event is exposure to a temperature of between about 36° C. and about 44° C. for a period of between about 0.5 minutes (min.) and about 30 min., at pH of between about 4.0 and about 7.8 (for oral tab forms), and wherein (ix) the cytoprotective pro-drug or the API is configured to have site-specific activity following activation by the activating agent.

In another exemplary implementation, provided herein is use of a pharmaceutical dispersion comprising: a dispersed phase comprising: a plurality of controlled-release microcapsules, micro-pellets, micro-beadles, micro-granules or mini tablets comprising a cytoprotective pro-drug or an active pharmaceutical agent (API), and an activating agent; and a continuous phase comprising a hydrocolloid matrix configured, upon exposure to a predetermined event, to allow contact between the plurality of controlled-release microcapsules comprising the cytoprotective pro-drug or the API, and the activating agent, in the process irradiating a tumor associated with: colorectal cancer, prostatic cancer, ovarian cancer, cervical cancer, or esophageal cancer, wherein (x) the cytoprotective pro-drug or active pharmaceutical agent comprises: ergotamine, amifostine, pyridoxine, or a composition comprising one or more of the foregoing and any permutation thereof, (xi) the cytoprotective pro-drug or active pharmaceutical agent comprises: S-2-(3-amino propylamino)ethyl dihydrogen phosphorothioate, its active 2-[(3-Aminopropyl)amino]ethanethiol dihydrochloride (WR-1065), their combination (at any permutation) and their pharmaceutically accepted salt, wherein (xii) the activating agent is: a protease, Oxyreductase, Carboxylesterase, Paraoxonase, Alkaline phosphatase, b-glucuronidase, Cytosine deaminase, or an activating agent composition comprising one or more of the foregoing, wherein (xiii) the pharmaceutical dispersion is in a suppository form, (xiv) in an injectable form, (xv) in an oral tab form, wherein (xvi) the predetermined event is exposure to a temperature of between about 36° C. and about 44° C. for a period of between about 3 minutes (min.) and about 30 min, or (xvii) the predetermined event is exposure to a temperature of between about 36° C. and about 44° C. for a period of between about 0.5 minutes (min.) and about 30 min., at pH of between about 4.0 and about 7.8 (for oral tab forms), and wherein (xviii) the cytoprotective pro-drug or the API is configured to have site-specific activity following activation by the activating agent.

While in the foregoing specification the pharmaceutical compositions, and their methods of use have been described in relation to certain preferred exemplary implementations, and many details are set forth for purpose of illustration, it will be apparent to those skilled in the art that the disclosure is susceptible to additional exemplary implementations and that certain of the details described in this specification and as are more fully delineated in the following claims can be varied considerably without departing from the basic principles of this invention.

1. A pharmaceutical dispersion comprising:
a) a dispersed phase comprising: a plurality of controlled-release microcapsules, micro-pellets, micro-beadles, micro-granules or mini tablets comprising a cytoprotective pro-drug or an active pharmaceutical agent (API), and an activating agent; and
b) a continuous phase comprising a hydrocolloid matrix configured, upon exposure to a predetermined event, to allow contact between the plurality of controlled-release microcapsules comprising the cytoprotective pro-drug or the active pharmaceutical agent, and the activating agent.

2. The pharmaceutical dispersion of claim 1, wherein the cytoprotective pro-drug or active pharmaceutical agent comprises: ergotamine, amifostine, pyridoxine, or a composition comprising one or more of the foregoing.

3. The pharmaceutical dispersion of claim 2, wherein the cytoprotective pro-drug or active pharmaceutical agent comprises: S-2-(3-amino propylamino) ethyl dihydrogen phosphorothioate, its active 2-[(3-Aminopropyl)amino]ethanethiol dihydrochloride (WR-1065), their combination and their pharmaceutically accepted salt.

4. The pharmaceutical dispersion of claim 1, wherein the activating agent is: a protease, Oxyreductase, Carboxylesterase, Paraoxonase, Alkaline phosphatase, β-glucuronidase, Cytosine deaminase, or an activating agent composition comprising one or more of the foregoing.

5. The pharmaceutical dispersion of claim 1, wherein the pharmaceutical dispersion is in a suppository form.

6. The pharmaceutical dispersion of claim 1, wherein the pharmaceutical dispersion is in an injectable form.

7. The pharmaceutical dispersion of claim 1, wherein the pharmaceutical dispersion is in an oral tab form.

8. The pharmaceutical dispersion of claim 4, wherein the predetermined event is exposure to a temperature of between about 36° C. and about 44° C. for a period of between about 3 minutes (min.) and about 30 min.

9. The pharmaceutical dispersion of claim 5, wherein the predetermined event is exposure to a temperature of between about 36° C. and about 44° C. for a period of between about 3 minutes (min.) and about 30 min.

10. The pharmaceutical dispersion of claim 6, wherein the predetermined event is exposure to a temperature of between about 36° C. and about 44° C. for a period of between about 3 minutes (min.) and about 30 min.

11. The pharmaceutical dispersion of claim 7, wherein the predetermined event is exposure to a temperature of between about 36° C. and about 44° C. for a period of between about 0.5 minutes (min.) and about 30 min., at pH of between about 4.0 and about 7.8.

12. The pharmaceutical dispersion of claim 1, wherein the cytoprotective pro-drug or the API is configured to have site-specific activity following activation by the activating agent.

13. Use of a pharmaceutical dispersion comprising: a dispersed phase comprising: a plurality of controlled-release microcapsules, micro-pellets, micro-beadles, micro-granules or mini tablets comprising a cytoprotective pro-drug or an active pharmaceutical agent (API), and an activating agent; and a continuous phase comprising a hydrocolloid matrix configured, upon exposure to a predetermined event, to allow contact between the plurality of controlled-release microcapsules comprising the cytoprotective pro-drug or the API, and the activating agent, in the process irradiating a tumor associated with: colorectal cancer, prostatic cancer, ovarian cancer, cervical cancer, or esophageal cancer.

14. The use of claim 13, wherein the cytoprotective pro-drug or active pharmaceutical agent comprises: ergotamine, amifostine, pyridoxine, or a composition comprising one or more of the foregoing.

15. The use of claim 14, wherein the cytoprotective pro-drug or active pharmaceutical agent comprises: S-2-(3-amino propylamino) ethyl dihydrogen phosphorothioate, its active 2-[(3-Aminopropyl) amino]ethanethiol dihydrochloride (WR-1065), their combination and their pharmaceutically accepted salt.

16. The use of claim 13, wherein the activating agent is: a protease, Oxyreductase, Carboxylesterase, Paraoxonase, Alkaline phosphatase, β-glucuronidase, Cytosine deaminase, or an activating agent composition comprising one or more of the foregoing.

17. The use of claim 13, wherein the pharmaceutical dispersion is in a suppository form.

18. The use of claim 13, wherein the pharmaceutical dispersion is in an injectable form.

19. The use of claim 13, wherein the pharmaceutical dispersion is in an oral tab form.

20. The use of claim 16, wherein the predetermined event is exposure to a temperature of between about 36° C. and about 44° C. for a period of between about 3 minutes (min.) and about 30 min.

21. The use of claim 17, wherein the predetermined event is exposure to a temperature of between about 36° C. and about 44° C. for a period of between about 3 minutes (min.) and about 30 min.

22. The pharmaceutical dispersion of claim 18, wherein the predetermined event is exposure to a temperature of between about 36° C. and about 44° C. for a period of between about 3 minutes (min.) and about 30 min.

23. The pharmaceutical dispersion of claim 19, wherein the predetermined event is exposure to a temperature of between about 36° C. and about 44° C. for a period of between about 0.5 minutes (min.) and about 30 min., at pH of between about 4.0 and about 7.8.

24. The use of claim 13, wherein the cytoprotective pro-drug or the API is configured to have site-specific activity following activation by the activating agent.
